(19) United States
(12) Patent Application Publication
(10) Pub. No.: US 2013/0040915 A1
(43) Pub. Date: Feb. 14, 2013

(54) PHARMACEUTICAL COMPOSITION

(71) Applicants: José Lucio Nuñez, Parana - Entre Rios (AR); José Bernardo Iturraspe, Santa Fe (AR); Nora Adriana Moyano De Iturraspe, Santa Fe (AR); José Castillo, Sant Fe (AR)

(72) Inventors: José Lucio Nuñez, Parana - Entre Rios (AR); José Bernardo Iturraspe, Santa Fe (AR); Nora Adriana Moyano De Iturraspe, Santa Fe (AR); José Castillo, Sant Fe (AR)

(73) Assignee: CAPITAL, BUSINESS Y GESTION DE FINANZAS, S.L., Madrid (ES)

(21) Appl. No.: 13/582,714

(22) PCT Filed: Mar. 3, 2011
(86) PCT No.: PCT/ES2011/070136

§ 371 Date: Oct. 25, 2012

(30) Foreign Application Priority Data
Mar. 5, 2010 (AR) P20100100664

(51) Int. Cl. A61K 31/675; A61P 35/00; A61J 1/00; A61P 19/08

(52) U.S. Cl. 514/94; 206/438

(57) ABSTRACT

An injectable pharmaceutical composition contained in a receptacle for injection into a human, which comprises a solution of zoledronic acid or the salts thereof, a solvent and a pH regulator.

### FIELD OF THE INVENTION

The present invention relates to injectable drugs containing bisphosphonates for treating normal and pathological bone resorption, particularly for treating certain cancers. Subcutaneously injectable zoledronic acid compositions are of special interest.

### STATE OF THE ART

Bisphosphonates have become an important therapy used in the treatment of cancer patients (Bloomfield D., J Clin Oncol. 1998; 16:1218-25, Should bisphosphonates be part of the standard therapy of patients with multiple myeloma or bone metastases from other cancers? An evidence-based review), among which can be mentioned clodronate, ibandronate, tiludronate, etidronate, alendronate, risedronate, and zoledronate. While their biological action is complex and not fully understood, they have been proven to be potent normal and pathological bone resorption inhibitors (Fleisch H., 3^{rd} Ed., New York, The Parthenon Publishing Group, 1997, Bisphosphonates in Bone Disease), particularly by controlling tumor-induced damage in bone resorption caused by osteoclasts (Body J. et al., J Clin Oncol. 1998; 16: 3890-9, Current use of bisphosphonates in oncology. International Bone and Cancer Study Group). It is the treatment of choice of tumor-induced hypercalcaemia (Purohit, O. et al., Br J. Cancer. 1995; 72: 1289-1293. A randomised double-blind comparison of intravenous pamidronate and clodronate in the hypercalcaemia of malignancy), and has additional clinical indications for acute metastatic bone treatment with pain that does not respond to opioids, the prevention of morbid bone events and the prevention of steroid-induced osteoporosis.

The administration routes of bisphosphonates for treating tumor-induced hypercalcaemia and acute bone pain in terminal patients that are used today have significant drawbacks. Conventional endovenous administration is a real drawback for non-hospitalized individuals treated on an outpatient basis due to the need for cannulation and subsequent hospital stay. On the other hand, the oral bioavailability of these bisphosphonates is too low and erratic (less than the 1%) and their gastrointestinal side effects in oral administration, such as esophagitis and esophageal ulcers, can prevent correctly complying with oral administration regimens which would naturally be preferred (Major, P. et al., Cancer, 2000; 88:6-14, Oral bisphosphonates: A review of clinical use in patients with bone metastases). Oral route administration in the patients with nausea is inappropriate.

The subcutaneous bolus administration of bisphosphonate salts in clinically active doses is problematic because they cause inflammation, pain and necrosis at the injection site which do not occur with endovenous administration, as mentioned in U.S. Pat. No. 4,446,052A, column 1, paragraph 25. Said patent solves the problem for a bisphosphonate salt that is not zoledronic acid by formulating a subcutaneous injection in gel form.

Administration by subcutaneous infusion of clodronate, a known bisphosphonate, has proven to be well tolerated and effective for controlling tumor-induced hypercalcaemia and has allowed solving the aforementioned drawbacks by means of the new administration by subcutaneous infusion, (Roemer-Bécuwe. C. et al., J. Pain Symptom Manage. 2003; 26:843-8, Safety of subcutaneous clodronate and efficacy in hypercalcaemia of malignancy: a novel route of administration). Roemer-Bécuwe administered 1500 mg of clodronate dissolved between 50 and 1000 ml of 5% dextrose or normal saline solution, in an infusion time of 2 to 24 hours, with a 23G×¾″ needle by means of gravitational pressure (without an infusion pump) in patients with bone pain or osteoporosis. In this original scientific paper, although complete consensus concerning the time, volume and site of administration of the subcutaneous infusion is not reached, the therapy therein is proven to be effective in lowering the level of calcium in the 149 patients treated with 254 subcutaneous infusions.

Zoledronic acid is one of the current bisphosphonates of choice given its therapeutic action and safety (Green, J. et al., Drug Development Research, 2002; 55: 210-224, Pharmacologic Profile of Zoledronic Acid a Highly Potent Inhibitor of Bone Resorption).

Zoledronic acid in Zometa® 4 mg/5 ml in aqueous solution with pH 6.5 has 24 mg of sodium citrate USP as a buffering agent and 220 mg of mannitol USP as a neutral excipient, suitable for completing its isotonicity. This is a formulation that has been designed for administration by intravenous infusion and must be applied diluted in dextrose or normal saline solution, such that by gravitational dripping administration can be completed in not less than 15 minutes. In the case of Aclasta®, which has 5 mg diluted in 100 ml of a ready-to-use solution for intravenous infusion, the formulation and recommended infusion time thereof is similar to that of Zometa® in an equivalent dilution.

The patient information leaflet for Zometa® reports that it has been used subcutaneously in female rats, pregnant female rats and pregnant female rabbits, without specifying the formulation used. The volume of the Zometa 5 ml formulation, the high concentration of citrate (4.8 mg/ml) and a pH of 6.5 are factors which advise against using this formulation by subcutaneous route in bolus in humans, from the pharmacotechnical perspective.

The presence of sodium citrate at high concentrations, of the order of 4.8 mg/ml, as presented by the Zometa® formulation, causes more pain after a subcutaneous injection than a solution with histidine (Laursen T. et al., Basic Clin. Pharmacol. Toxicol., 2006; 98: 218-21, Pain perception after subcutaneous injections of media containing different buffers). Although citrate is used in subcutaneous formulations, the standard concentrations are much lower than those present in the Zometa® formulation.

The Zometa® and Aclasta® formulations are sterilized in their final form by autoclave to confer to them greater microbiological safety than a simple aseptic manufacture does because zoledronate is chemically very stable in that autoclave sterilization condition. Zoledronic acid at pH 6.5 when it is in aqueous solution and in typical sterilization temperature and pressure conditions (temperature range of 121 to 135° C., pressure range of 2 to 3 atm and sterilization time range of 20 to 45 min) has proven to be incompatible with type I borosilicate, given that it reacts with divalent alkaline-earth metals, such as calcium, magnesium, iron and barium and trivalent alkaline-earth metals such as aluminum and iron, which has the natural composition of type I glass, to form salts with zoledronic acid that are very insoluble in water, making the inner surface of the glass in contact with the aqueous solution of the active ingredient during sterilization opaque, indicating that this type of glass is unsuitable.

Patent application WO 2005025551 of Novartis claims to solve the problem of the incompatibility of bisphosphonates with glass using bottles made of plastic materials. Said bottles are washed and sterilized by autoclave prior to use. The plastic bottles for injectable products sterilized by autoclave have two particular problems, first of all they have a high level of organic extractables allowed, and second of all the deformability of the plastic at the product sterilization temperature in an autoclave due to the presence of air inside the vial, which has a high internal pressure with respect to the external pressure (in aqueous solutions without organic solvents, this occurs due to the thermal expansion of the air contained in the vial), after being autoclaved, causing the aluminum seals to become loose and not hermetically sealed when they are cooled at room temperature in a proportion that can exceed 10% of the vials of a production batch, so they must be discarded when controlled for final approval.

The bottles made of glass have better mechanical properties than plastics suitable for manufacturing injectable products. After the autoclaving process, the vial made of glass closed with a stopper and sealed with aluminum has proven leak-tightness. On the other hand, the level of organic extractables is lower compared to the plastic material.

Patent application WO 0222136 A1 of Faulding, claims solving the aforementioned problem by using bottles made of type I glass specially treated with silicones to obtain an internal polymer film.

There have been various attempts to obtain a subcutaneous bisphosphonate formulation. Some of them are cited below:

Patent ES2254410 claims a gel type pharmaceutical composition for the subcutaneous administration of bisphosphonates characterized by having a compound which reduces the diffusion of the bisphosphonate into the tissue, which is a natural polymer (cellulose derivatives, alginic acid derivatives, alginic acid-dextran derivatives, gelatins, collagen, hyaluronic acid, dermatan, and heparan sulfate) and calcium ions.

Patent PCT/US2009/002353 claims the use of a subcutaneous bisphosphonate formulation, containing hyaluronidase to reduce damage at the injection site, an enzyme of known therapeutic use which hydrolyzes hyaluronic acid of the subcutaneous connective tissue to favor its passive diffusion into subcutaneous tissue and improve dermal absorption.

Patent DE 4244422 claims the use of insoluble zinc and magnesium salts of bisphosphonates to be used as a prolonged release suspension for subcutaneous or intramuscular administration.

Patent EP 0449405 claims an aqueous suspension of the monocalcium salt of alendronic acid which reduces tissue damage, pain and irritation after intramuscular or subcutaneous administration.

U.S. Pat. No. 4,446,052 claims an aqueous gel containing the tricalcium salt of di(1-hydroxy-3-aminopropane-1,1-diphosphonic) acid which provides a slow a release of the active ingredient, reducing damage at the injection site.

Patent JP5238929 is a pharmaceutical preparation capable of releasing medicinal substances at a constant rate, including bisphosphonates, suitable for subcutaneous implantation, preferably as a hollow capsule.

U.S. Pat. No. 5,662,918 claims the use of polyethylene glycol to prevent the bisphosphonates from reacting with metals of the glass at a pH between 3 and 4.5.

U.S. Pat. No. 7,090,865 is a composition and method for treating anemia, with a composition comprising bisphosphonates and a suitable carrier.

Bisphosphonates are used in the state of the art for treating osteoporosis, acting biologically after being absorbed by the bones, where they accumulate for a long time while they act by inhibiting normal bone resorption and preventing the action of the osteoclasts, to then be slowly released from the bone to finally be eliminated by renal route.

Some of the drawbacks of the current intravenous dosage of zoledronic acid are:

It requires patients having to stay temporarily in hospital.
It requires nursing staff trained specifically for said administration.
Increase of cost per hour of the medical-nursing-administrative system.
Increase of cost due to the use of consumables (IV lines, catheters, saline, syringes).
Increase of cost by billing the public healthcare system.

Despite these drawbacks, there are no injectable subcutaneous zoledronic acid formulations on the market. Some of the technological attempts to obtain them have been mentioned above though none of them has achieved the expected success.

The present invention solves the technical problems raised in the state of the art.

The present invention provides a pharmaceutical composition contained in a container for being injected in a human which, being injected by subcutaneous route, has a pharmacokinetics equivalent to that caused by an intravenous infusion, such as that used as the standard prescription today.

The pharmaceutical composition of the present invention allows:

Not requiring patients to have to stay temporarily in hospital.
Not requiring nursing staff trained specifically for said administration.
Not increasing the cost per hour of the medical-nursing-administrative system.
Not increasing the cost due to the use of consumables (IV lines, catheters, saline, syringes).
Not increasing the cost by billing the public healthcare system.
Use of an administration route that is less aggressive for the patient.
Easy administration and probability of self-administration.
Possibility of administering increasing doses.

### BRIEF DESCRIPTION OF THE INVENTION

The present invention provides a pharmaceutical composition contained in a container for being injected in a human comprising a solution of zoledronic acid or its salts, a solvent and a pH regulator. Said pharmaceutical composition preferably comprises said zoledronic acid at a concentration between 0.05 and 50 mg/ml, preferably between 0.5 and 25 mg/ml, more preferably between 4 and 16 mg/ml.

In a preferred embodiment, the present invention provides a pharmaceutical composition contained in a container for being injected in a human comprising an isotonic solution of zoledronic acid or its salts, a solvent, a pH regulator and further comprising a thickener selected from the group comprised by albumin and PEG.

In a preferred embodiment, the present invention provides a pharmaceutical composition contained in a container for being injected in a human comprising an isotonic solution of zoledronic acid or its salts, a solvent, a pH regulator and further comprising a surfactant selected from the group comprised by sodium taurodeoxycholate, sodium taurocholate, sodium cholate, sodium deoxycholate, macrogol 15 hydroxystearate (Solutol SH-15), sorbitan monolaurate and Tween or their mixtures, preferably at a concentration between 0.01 and 1% w/v.

In a preferred embodiment, the present invention provides a pharmaceutical composition contained in a container for being injected in a human comprising a solution of zoledronic acid or its salts, a solvent, a pH regulator and further comprising a vasodilator selected from the group comprised by acetylcholine chloride and adenosine triphosphate, or their mixtures, preferably at a concentration between 0.01 and 2% w/v.

In a preferred embodiment, the present invention provides a pharmaceutical composition contained in a container for being injected in a human comprising a solution of zoledronic acid or its salts, a solvent, a pH regulator and further comprising a neutral excipient selected from the group comprised by lactose, sorbitol, sucrose, mannitol and their mixtures, preferably at a concentration between 0.01 and 8% w/v.

In a preferred embodiment, the present invention provides a pharmaceutical composition contained in a container for being injected in a human comprising a solution of zoledronic acid or its salts, a solvent, a pH regulator and further comprising an adjuvant excipient selected from the group comprised by aluminum hydroxide, zinc acetate and their mixtures, preferably at a concentration between 0.01 and 1.8% w/v.

In a preferred embodiment, the present invention provides a pharmaceutical composition contained in a container for being injected in a human comprising a solution of zoledronic acid or its salts, a solvent, a pH regulator, a thickener selected from the group comprised by albumin and PEG, and a vasodilator selected from the group comprised by acetylcholine chloride and adenosine triphosphate, or their mixtures.

In a preferred embodiment, the present invention provides a pharmaceutical composition contained in a container for being injected in a human comprising a solution of zoledronic acid or its salts, a solvent, a pH regulator, a thickener selected from the group comprised by albumin and PEG, and a surfactant selected from the group comprised by sodium taurodeoxycholate, sodium taurocholate, sodium cholate, sodium deoxycholate, macrogol 15 hydroxystearate (Solutol SH-15), sorbitan monolaurate and Tween or their mixtures, and a neutral excipient selected from the group comprised by lactose, sorbitol, sucrose, mannitol and their mixtures.

In a preferred embodiment, said pharmaceutical composition is injected by subcutaneous route, i.e., it is a composition for subcutaneous injection. Said pharmaceutical composition further comprises a pH that is between 6 and 7.4. And said solvent is selected from the group comprised by water, ethanol, propylene glycol, glycerol, glycofurol, isopropyl alcohol, dimethylsulfoxide and their mixtures.

In a preferred embodiment, the present invention provides an isotonic pharmaceutical composition.

In a preferred embodiment of the present invention, said container is made of glass with an inner surface silicon dioxide film; said container is preferably made of improved type I glass sealed with a Teflon-coated bromobutyl stopper that can be sterilized by an autoclave.

In another embodiment of the invention said container is a syringe.

Said pharmaceutical composition of the invention comprises a total liquid volume between 0.1 and 100 ml, preferably between 0.2 and 5 ml, more preferably between 0.25 and 2 ml.

The zoledronic acid of the composition of the invention is preferably dissolved and not in the form of precipitate or in the form of a deposit inside a microcapsule or implant made of a biocompatible biopolymer.

In the pharmaceutical composition contained in a container for being injected of the present invention, said container is made of glass with an inner surface silicon dioxide film and closed with a leak-tight stopper; after being sterilized it is essentially free of extractables and said stopper continues to be hermetically sealed.

### DETAILED DESCRIPTION OF THE INVENTION

The present invention solves the pharmacotechnical problems raised for a subcutaneous injectable composition in bolus comprising zoledronic acid without the need for it to be a gel.

In a preferred embodiment, the present invention is a pharmaceutical composition that is injected under the dermis and before hitting the muscle, being sterile, pyrogen free, and having a volume not less than 0.2 ml and not greater than 2 ml, particularly between 0.25 to 1.0 ml, because the pain of a subcutaneous injection is related to the injection volume and the application site. The study conducted by Jorgensen, J. et al.; Ann. Pharmacother., 1996; 30: 729-32, Pain assessment of subcutaneous injections, indicates that increasing the volume from 0.5 to 1.0 ml causes a significant increase in pain. Its excipients and solvents furthermore do not interfere with the efficacy of zoledronic acid. The present invention is a preferably aqueous pharmaceutical composition that is manufactured by filling, closing and sterilizing the vials in an autoclave. However it can also be prepared in aseptic conditions without final sterilization, where the solution is sterilized by filtration and the vial is manufactured aseptically. This pharmaceutical composition in aqueous solution is more convenient than that of an emulsion, or suspension, ready to be used or prepared by means of reconstituting a sterile or lyophilized solid or mixing two sterile solutions which must meet the requirements of reproducibility, homogeneity, stability and injectability of the resuspension or emulsion, because the physicochemical stability of the solutions is less complicated and more predictable than that of suspensions and the emulsions. Furthermore the pharmaceutical composition of the present invention is biocompatible and isotonic.

Human albumin acts like a thickening cosolvent (Olson, W. et al., J. Parenter. Sci. Technol, 1988; 42: 82-5, Human serum albumin as a cosolvent for parenteral drugs).

Human albumin is obtained from human blood that has previously been heparinized and analyzed individually for the viruses recommended in international guidelines, thermally treated for ten hours at 60° C. to inactivate the hepatitis A, B and C viruses and HIV, with the prior addition of sodium caprylate or N-acetyltryptophan sodium as stabilizers to prevent denaturation, or preferably it is possible to use recombinant human albumin, as in the product Recombumin® by Novozymes Biopharma, or the GTC Biotherapeutics or Prospect products, to prevent the risk of transmitting new and known infectious agents (such as West Nile Virus and the agent causing Creutzfeldt-Jakob disease).

A preferred embodiment of the new subcutaneous injectable pharmaceutical composition in bolus comprising zoledronic acid of the invention contains human albumin and is biocompatible according to studies conducted in animals.

A preferred alternative of this new pharmaceutical composition of the invention has a pH between 6.0 and 7.4, with concentrations of sodium phosphates as a buffering agent from 5 to 50 mM, preferably 25 mM.

Another inventive aspect of the pharmaceutical composition claimed herein is that is that it comprises a vasodilating agent, such as acetylcholine chloride (as a parasympathomimetic agent: Martindale, The Extra Pharmacopoeia, 29th Ed., The Pharmaceutical Press, 1989, p. 1328) or adenosine triphosphate (Bojórn Folkow Acta Physiologica Scandinavica, 1949; 17: 311-316. The Vasodilator Action of Adenosine Triphosphate; Hendrik J. Agteresch et al. Drugs, 1999; 58: 211-232. Adenosine Triphosphate: Established and Potential Clinical Applications; Martindale, The Extra Pharmacopoeia, 29th Ed., The Pharmaceutical Press, 1989, p. 1392), in its sodium form, at a small mass ratio that is enough for it to act locally as a vasodilator at the site and time of the injection, but which is not enough to cause a systemic vasodilator clinical action. Subcutaneous absorption of zoledronic acid is thereby improved, and its formulation becomes more biocompatible, reducing known cutaneous necrosis problems.

This new subcutaneous injectable pharmaceutical composition in bolus comprising zoledronic acid of the invention has a pH comprised between 6.0 and 7.4, has a zoledronic acid concentration comprised between 0.05 and 50 mg/ml, preferably between 0.5 and 25 mg and more specifically from 4 to 16 mg/ml, optionally ionic surfactant agents at concentrations of 0.01 at 1% w/v, such as sodium taurocholate, sodium taurodeoxycholate, sodium cholate, sodium deoxycholate, and or non-ionic surfactant agents at concentrations of 0.0.1. at 1% w/v, such as macrogol 15 hydroxystearate (Solutol SH-15), sorbitan monolaurate, pH regulating agents at concentrations of 0.01 at 0.5% w/v, such as sodium hydroxide, sodium phosphate, sodium citrate, and sodium lactate, optionally neutral excipients at concentrations of 0.01 at 8% w/v, such as lactose, sorbitol, sucrose and mannitol, solvents such as water, ethanol, propylene glycol, glycerol, glycofurol, isopropyl alcohol, dimethylsulfoxide and their mixtures, optionally thickening agents at concentrations of 0.01 at 5% w/v, such as human albumin, polyethylene glycol (PEG 3350), optionally adjuvant excipients at concentrations of 0.01 at 8% w/v, such as aluminum hydroxide, zinc acetate, optionally subcutaneous acting vasodilators at concentrations of 0.01 at 2% w/v, such as acetylcholine chloride and adenosine triphosphate, in their sodium or acid salts, in all possible combinations, for the purpose of obtaining an isotonic aqueous formulation that is biocompatible with the human epidermis and dermis, with a total final volume of 0.1 to 100 ml, preferably 0.2 to 5 ml and more preferably 0.25 to 2 ml, and the use of a vial having a suitable volume made of improved type I glass, or a plastic syringe so that it does not interfere with the zoledronic acid.

Sodium deoxycholate at concentrations of 1% works as a surfactant causing the non-specific rupture of adipose tissue cell membranes (Dermatol Surg. 2004; 30:1001-8. Detergent effects of sodium deoxycholate are a major feature of an injectable phosphatidylcholine formulation used for localized fat dissolution. Rotunda, A M et al.).

The new subcutaneous injectable pharmaceutical composition in bolus comprising zoledronic acid of the invention can comprise the presence of sodium taurocholate (pKa 1.4) or sodium taurodeoxycholate as an ionic surfactant suitable for favoring the passive diffusion of the active ingredient, which is more appropriate than sodium deoxycholate (pKa 6.58) because this carboxylate (deoxycholate) requires a pH higher than the 7.4 of the skin (it is sparingly soluble at that pH) to work properly as a surfactant, whereas the detergent action of taurocholic acid or taurodeoxycholic acid, which are sulfonic acids, at that biological pH is optimal.

The present invention can be suitably administered by subcutaneous route in bolus, being biocompatible in animal studies.

The vial containing this novel pharmaceutical composition is a vial made of improved type I borosilicate which has an inner surface silicon dioxide film. The vial produced by a plasma-enhanced chemical vapor deposition process by means of depositing the silicon dioxide in gas phase on the glass at a high temperature producing a thin layer of silicon dioxide (100 to 200 nm) which prevents the normal diffusion of alkaline metals and allows this new aqueous pharmaceutical composition to be stable, even in autoclaving conditions, due to the absence of alkaline metals on the walls in contact with the zoledronic acid solution during its extended shelf life period is preferred. The improved type I bottle of this novel form allows applying standard material washing, depyrogenation and sterilization techniques, requiring no special equipment or processes for conditioning and use, as in the manufacture of injectable products in plastic vials.

The polyethylene glycol of the present invention is used as a thickening agent, at a pH comprised between 6.0 and 7.4 and in a vial made of improved type I glass. It is not used to prevent the bisphosphonates from reacting with the metals of the glass at a pH between 3 and 4.5, as described in U.S. Pat. No. 5,662,918, because the vial made of improved type I borosilicate does not present this problem.

In a preferred embodiment of the invention, said injectable pharmaceutical composition comprising zoledronic acid is packaged in a container formed by a syringe, preferably in a plastic syringe of those known as pre-filled syringes. Packaging the pharmaceutical composition of the invention in ready-to-apply plastic syringes requires the product to be prepared in aseptic conditions, achieving sterility by means of filtration, because said syringes do not withstand sterilization using heat. This alternative has the advantage of preventing handling, reducing error in dosing the preparation of the injection of 0.25 to 0.5 ml with small 27 to 32 G gauge needles, for reducing the pain at the application site.

### APPLICATION EXAMPLES

### Example 1

2.13 g of zoledronic acid monohydrate corresponding to 2.00 g of zoledronic acid anhydrous (MW 272.09 g/mol, C₅H₁₀N₂O₇P₂) are weighed, decanted into a 500 ml beaker and dissolved in 200 ml in water for injection alkalinized with 12.3 mmol of sodium hydroxide, to obtain a sodium zoledronate solution at pH 6.5 which is controlled with a pH meter at 20° C., where a value of 6.44 is obtained. 85 mg of sodium phosphate monobasic dihydrate MW 155.98 and 1015 mg of sodium phosphate dibasic dodecahydrate MW 177.96 are added and dissolved with stirring. 4.5 g of human albumin are added, the pH is taken to 7.0 with 1 N sodium hydroxide solution and is completed with water for injection at a total volume of 250 ml.

The sodium zoledronate solution is sterilized by filtration in a 0.22 micron nylon membrane by means of standard technique (controlling membrane integrity by a bubble-point test after filtering).

0.6 ml of the zoledronate solution are metered in aseptic conditions into bottles made of improved type I glass, the bottles are closed with a Teflon-coated bromobutyl stopper and sealed with an aluminum seal and plastic cover.

The following pharmaceutical composition was thereby obtained:

Zoledronic acid 4 mg
Human albumin 9 mg
Sodium phosphate monobasic dihydrate 0.17 mg
Sodium phosphate dibasic dodecahydrate 2.03 mg
NaOH to obtain pH 7.0

### WFI, qsf 0.5 ml.

The titer, strength, pH, vial leak-tightness, appearance (solution clarity and color), sterility and bacterial endotoxins were determined for the injectable product obtained.

The vials passed the leak-tightness test, neither cloudiness nor opalescence was observed in any vial, pH (7.0) did not vary with respect to the value found in the solution before autoclave sterilization, and all other tests complied with product specifications.

### Example 2

2.13 g of zoledronic acid monohydrate, corresponding to 2.00 g of zoledronic acid anhydrous (MW 272.09 g/mol, C₅H₁₀N₂O₇P₂) are weighed, decanted into a 1000 ml beaker and dissolved in 200 ml in water for injection which has 12.3 mmol of sodium hydroxide dissolved therein, to obtain a sodium zoledronate solution at pH 6.5 which is controlled with a pH meter at 20° C., where a value of 6.44 is obtained. 85 mg of sodium phosphate monobasic dihydrate MW 155.98 and 1015 mg of sodium phosphate dibasic dodecahydrate MW 177.96 are added and dissolved with stirring. 4.0 g of human albumin are added, 0.28 g of adenosine disodium triphosphate (98% dry base and with 9.4% moisture content by KF, from China) are added, the pH is taken to 7.0 with 1 N sodium hydroxide solution and is completed with water for injection at a total volume of 250 ml.

The sodium zoledronate solution is sterilized by filtration in a 0.22 micron nylon membrane by means of standard technique (controlling membrane integrity by a bubble-point test after filtering).

0.6 ml of the zoledronate solution are metered in aseptic conditions into bottles made of improved type I glass, the bottles are closed with a Teflon-coated bromobutyl stopper and sealed with aluminum and a plastic cover.

The following pharmaceutical composition was thereby obtained:

Zoledronic acid 4 mg
Adenosine triphosphate (as disodium salt) 0.5 mg
Human albumin 8 mg
Sodium phosphate monobasic dihydrate 0.17 mg
Sodium phosphate dibasic dodecahydrate 2.03 mg
NaOH to obtain pH 7.0

### WFI, qsf 0.5 ml.

The titer, strength, pH, vial leak-tightness, sterility and bacterial endotoxins were determined for the injectable product obtained.

The vials passed the leak-tightness test, neither cloudiness nor opalescence was observed in any vial, pH (7.0) did not vary with respect to the value found in the solution before autoclave sterilization, and all other tests complied with product specifications.

### Example 3

A solution with 50 mg of sodium taurodeoxycholate MW 503.69 (from www.nzp.co.nz) 125 mg of PEG 3350, 750 mg of mannitol (MW 182.17 g/mol, 213 mg of zoledronic acid monohydrate (equivalent to 200 mg of zoledronic acid) in 20 ml of water for injection is prepared and is taken to pH 6.5 with a 1 N sodium hydroxide solution, while it is dissolved by heating in a water bath with stirring and is completed with water for injection such that it has a total volume of 25 ml; it is filtered by 0.22 μm nylon membrane and 0.6 ml of the sterile solution were metered into 2 ml vials made of improved type I glass with a 13 mm opening. Once filled with the solution, the vials were stoppered with Teflon-coated bromobutyl stoppers sterilized by autoclave, sealed with aluminum seals with a plastic cover, and sterilized in an autoclave for 40 minutes at 121° C.

The following pharmaceutical composition was thereby obtained:

Zoledronic acid 4 mg
Sodium taurodeoxycholate 1 mg
Polyethylene glycol 3350 2.5 mg

### Mannitol 15 mg

NaOH to obtain pH 6.5

### WFI, qsf 0.5 ml.

The titer, strength, pH, vial leak-tightness, appearance (solution clarity and color), sterility and bacterial endotoxins were determined for the injectable product obtained.

The vials passed the leak-tightness test, neither cloudiness nor opalescence was observed in any vial, pH (6.5) did not vary with respect to the value found in the solution before autoclave sterilization, and all other tests complied with product specifications.

### Example 4

A solution with 50 mg of sodium taurodeoxycholate MW 503.69 (from www.nzp.co.nz), 125 mg of PEG 3350, 750 mg of mannitol (MW 182.17 g/mol), 213 mg of zoledronic acid monohydrate (equivalent to 200 mg of zoledronic acid) in 20 ml of water for injection is prepared and is taken to pH 7.0 with a 1 N sodium hydroxide solution, while it is dissolved by heating in a water bath with stirring and is completed with water for injection such that it has a total volume of 25 ml; it is filtered by 0.22 μm nylon membrane and 0.6 ml of the sterile solution were metered into 2 ml vials made of improved type I glass with a 13 mm opening. Once filled with the solution, the vials were stoppered with Teflon-coated bromobutyl stoppers sterilized by autoclave, sealed with aluminum seals with a plastic cover, and sterilized in an autoclave for 40 minutes at 121° C.

The following pharmaceutical composition was thereby obtained:

Zoledronic acid 4 mg

### Acetylcholine Chloride 1 mg

Sodium taurodeoxycholate 1 mg
Polyethylene glycol 3350 2.5 mg

### Mannitol 15 mg

NaOH to obtain pH 7.0

### WFI, qsf 0.5 ml

The titer, strength, pH, vial leak-tightness, appearance (solution clarity and color), sterility and bacterial endotoxins were determined for the injectable product obtained.

The vials passed the leak-tightness test, neither cloudiness nor opalescence was observed in any vial, pH (7.0) did not vary with respect to the value found in the solution before autoclave sterilization, and all other tests complied with product specifications.

### Example 5

0.7 liters of water for injection at 35° C. were placed in a stainless steel container having a suitable volume provided with stirring, and 3.6 g of sodium citrate USP and 32.6 g of mannitol USP were added with constant stirring. Stirring was maintained until complete dissolution for 30 minutes. It was cooled at room temperature. 633 mg of zoledronic acid monohydrate were added, dispersing it with permanent stirring for 30 minutes in a nitrogen atmosphere until dissolution. The pH was measured and it was verified that it was 6.3 and the necessary amount of injectable quality water was added until completing the final volume of 0.742 liters, stirring for 5 minutes until homogenizing. The solution was filtered by a 0.22 μm membrane for the purpose of sterilizing it freeing the solution from the presence of any foreign body. The sterile solution 5.3±0.1 ml was metered into vials made of improved type I glass, which vials were, once filled with the solution, stoppered with sterilized Teflon-coated bromobutyl stoppers, sealed with aluminum seals with a plastic cover, and sterilized in an autoclave for 40 minutes at 121° C. The product obtained was analyzed to determine its titer, strength, pH, vial leak-tightness, appearance (clarity and color of the solution), sterility and bacterial endotoxins. The vials passed the leak-tightness test, neither cloudiness nor opalescence was observed in any vial, pH (6.3) did not vary with respect to the value found in the solution before autoclave sterilization, and all other tests complied with product specifications.

The following pharmaceutical composition was thereby obtained:

Zoledronic acid 4 mg
Sodium citrate (trisodium dihydrate) 24 mg

### Mannitol 220 mg

### WFI, qsf 5.0 ml.

### Example 6

80 ml of water for injection at 35° C. were placed in a 250 ml beaker provided with magnetic stirring, and 110 mg of adenosine trisodium phosphate (98% dry base and with 9.4% moisture content by KF, from China), 309 mg of sodium citrate USP, 3012 mg of mannitol USP and 2.4 ml of a 1 N of NaOH solution were added with constant stirring. Stirring was maintained until complete dissolution for 30 minutes. It was cooled at room temperature. 430 mg of zoledronic acid monohydrate equivalent to 404 mg of zoledronic acid anhydrous were added, dispersing it with permanent stirring for 30 minutes in nitrogen bubbling until dissolution. It was taken to pH 6.9 with a 1 N NaOH solution and decanted into a 100 ml flask, and the necessary amount of injectable quality water was added until completing its total volume, stirring until homogenizing. The solution was filtered by a 0.22 μm membrane for the purpose of sterilizing it, freeing the solution from the presence of any foreign body. 1.1 ml of the sterile solution was metered into 2 ml vials made of improved type I glass. Once filled with the solution, the vials were stoppered with sterilized 13 mm West Teflon-coated bromobutyl stoppers, were sealed with aluminum seals with a plastic cover, and sterilized in an autoclave for 40 minutes at 121° C. The product obtained was analyzed to determine its titer, strength, pH, vial leak-tightness, sterility and bacterial endotoxins. The vials passed the leak-tightness test, neither cloudiness nor opalescence was observed in any vial, pH (6.9) did not vary with respect to the value found in the solution before autoclave sterilization, and all other tests complied with product specifications.

The following pharmaceutical composition was thereby obtained:

Zoledronic acid 4 mg
Adenosine trisodium phosphate 1 mg
Sodium citrate (trisodium dihydrate) 3 mg

### Mannitol 30 mg

NaOH to obtain pH 7.0

### WFI, qsf 1.0 ml.

In Examples 1 to 6 the 13 mm West Teflon-coated bromobutyl stoppers used in the experiments were sterilized in an autoclave for 40 minutes at 121° C.

The 2 ml bottles made of improved type I glass with a 13 mm opening were washed according to standard validated procedures in the automatic washer with water for injection at 70-75° C. and were then depyrogenated and sterilized using drying heat in an oven for 180 minutes at 220° C.

Human albumin: the necessary volume of 20% w/v aqueous solution from the Blood Derivative Laboratory “Presidente Illia”, UNC, Córdoba, Argentina, was used.

The leak-tightness of the vials filled with the composition of the invention was determined with methylene blue according to European Pharmacopeia 6.0 method 3.2.9, pp. 386-7.

### Example 7

HPLC analysis of the zoledronic acid formulations (method based on the paper by Oscar Quattrocchi et al., Journal of Pharmaceutical and Biomedical Analysis. 2001, 24, 1011-1018, Ion exchange chromatographic determination of olpadronate, phosphate, phosphite, chloride and methanesulfonic acid):

A Waters HPLC equipment was used that was equipped with a binary model 1525 pump, Waters 2996 diode array detector, model 717 plus automatic injector, with Empower Software, a 5 μm C18 column, 25 cm×4.6 mm (Zorbax SB from Agilent Technologies, Inc., Palo High, Calif.), placed in an oven at 40° C., injecting 10 μl of a zoledronic solution at 1 mg/ml, with a 75:25 buffer/acetonitrile mixture, at a flow rate of 1 ml/min, with a 220 nm UV detector (buffer: 3 g of lauryl sulfate sodium in one liter of distilled water, adding 40 ml of concentrated phosphoric acid and taken to pH 1 with an aqueous solution of perchloric acid at 70%, with zoledronic eluting at 2.7 minutes, imidazole between 10 and 15 minutes, and imidazole acetic acid between 6 to 10 minutes

According to the experiments conducted, the results of which are reported, the sterilized formulations of Examples 1 to 6 have a purity similar to the raw material used and the initial titer is not altered either.

### Pharmaceutical Composition of Example 1

Zoledronic acid 4 mg
Human albumin 9 mg
Sodium phosphate monobasic dihydrate 0.17 mg
Sodium phosphate dibasic dodecahydrate 2.03 mg
NaOH to obtain pH 7.0

### WFI, qsf 0.5 ml.

### Blank of Pharmaceutical Composition of Example 1

Human albumin 9 mg
Sodium phosphate monobasic dihydrate 0.17 mg
Sodium phosphate dibasic dodecahydrate 2.03 mg
NaOH to obtain pH 7.0

### WFI, qsf 0.5 ml.

TABLE 1
PURITY BY HPLC- PHARMACEUTICAL COMPOSITION OF EXAMPLE 1

| | | | | | | % Relative | |
|---|---|---|---|---|---|---|---|
| | | 1^{st} | | | % Average | Standard | Total |
| | | Measurement | Duplicate | Triplicate | Impurities | Deviation | Impurities |
| Zoledronic Acid | Retention | 2.606 | 2.696 | 2.700 | | | |
| | Time (RT) | | | | | | |
| Imidazole | % Area | 0 | 0 | 0 | 0.00 | — | NA |
| Acetic Acid | | | | | | | |
| | Retention | — | — | — | — | — | NA |
| | Time (RT) | | | | | | |
| | Relative | — | — | — | — | — | NA |
| | Retention | | | | | | |
| | Time | | | | | | |
| Imidazole | % Area | 0.09 | 0.10 | 0.10 | 0.10 | 5.97 | NA |
| | Retention | 10.835 | 10.877 | 10.868 | 10.860 | 0.20 | NA |
| | Time (RT) | | | | | | |
| | Relative | 4.158 | 4.034 | 4.025 | 4.072 | 1.82 | NA |
| | Retention | | | | | | |
| | Time | | | | | | |
| Max. Individual | % Area | 0.02 | 0.04 | 0.03 | 0.03 | 33.33 | NA |
| Impurity | | | | | | | |
| | Retention | 10.296 | 10.306 | 10.259 | 10.287 | 0.24 | NA |
| | Time (RT) | | | | | | |
| | Relative | 3.951 | 3.823 | 3.800 | 3.858 | 2.11 | NA |
| | Retention | | | | | | |
| | Time | | | | | | |

| | | | | | | |
|---|---|---|---|---|---|---|
| Chromatographic Purity | 99.89 | 99.86 | 99.87 | 99.97 | 0.02 | 0.13 |

Summary of Results

| Imidazole | | Max. Indiv. | Total |
|---|---|---|---|
| Acetic Acid | Imidazole | Impurity | Impurities |
| 0.00 | 0.10 | 0.03 | 0.13 |
| Max. 0.5% | Max. 0.5% | Max. 1.0% | Max. 2.5% |

NA: Does not apply

### Pharmaceutical Composition of Example 3

Zoledronic acid 4 mg
Sodium taurodeoxycholate 1 mg
Polyethylene glycol 3350 2.5 mg

### Mannitol 15 mg

NaOH to obtain pH 7.0

### WFI, qsf 0.5 ml.

### Blank of Pharmaceutical Composition of Example 3

Sodium taurodeoxycholate 1 mg
Polyethylene glycol 3350 2.5 mg

### Mannitol 15 mg

NaOH to obtain pH 7.0

### WFI, qsf 0.5 ml.

TABLE II
PURITY BY HPLC- PHARMACEUTICAL COMPOSITION OF EXAMPLE 3

| | | | | | | % Relative | |
|---|---|---|---|---|---|---|---|
| | | 1^{st} | | | % Average | Standard | Total |
| | | Measurement | Duplicate | Triplicate | Impurities | Deviation | Impurities |
| Zoledronic Acid | Retention | 2.689 | 2.666 | 2.678 | | | |
| | Time (RT) | | | | | | |
| Imidazole | % Area | 0 | 0 | 0 | 0.00 | — | NA |
| Acetic Acid | | | | | | | |
| | Retention | — | — | — | — | — | NA |
| | Time (RT) | | | | | | |
| | Relative | — | — | — | — | — | NA |
| | Retention | | | | | | |
| | Time | | | | | | |
| Imidazole | % Area | 0 | 0 | 0 | 0.00 | — | NA |
| | Retention | — | — | — | — | — | NA |
| | Time (RT) | | | | | | |
| | Relative | — | — | — | — | — | NA |
| | Retention | | | | | | |
| | Time | | | | | | |
| Max. Individual | % Area | 0.09 | 0.06 | 0.07 | 0.07 | 20.83 | NA |
| Impurity | | | | | | | |
| | Retention | 4.151 | 4.13 | 4.142 | 4.141 | 0.25 | NA |
| | Time (RT) | | | | | | |
| | Relative | 1.544 | 1.549 | 1.547 | 1.547 | 0.18 | NA |
| | Retention | | | | | | |
| | Time | | | | | | |
| Unidentified | % Area | 0.06 | 0.06 | 0.06 | 0.06 | 0.00 | NA |
| Peak | | | | | | | |
| | Retention | 4.849 | 4.821 | 4.826 | 4.832 | 0.31 | NA |
| | Time (RT) | | | | | | |
| | Relative | 1.803 | 1.808 | 1.802 | 1.805 | 0.18 | NA |
| | Retention | | | | | | |
| | Time | | | | | | |

| | | | | | | |
|---|---|---|---|---|---|---|
| Chromatographic purity | 99.72 | 99.88 | 99.87 | 99.82 | 0.09 | 0.18 |

Summary of Results

| Imidazole | | Max. Indiv. | Total |
|---|---|---|---|
| Acetic Acid | Imidazole | Impurity | Impurities |
| 0.00 | 0.00 | 0.07 | 0.18 |
| Max. 0.5% | Max. 0.5% | Max. 1.0% | Max. 2.5% |

NA: Does not apply

1. An injectable pharmaceutical composition contained in a container, characterized in that it comprises a solution of zoledronic acid or its salts, a solvent and a pH regulator.

2. The pharmaceutical composition according to claim 1, characterized in that it is injectable by subcutaneous route.

3. The pharmaceutical composition according to claim 1, characterized in that it comprises said zoledronic acid at a concentration between 0.05 and 50 mg/ml.

4. The pharmaceutical composition according to claim 1, characterized in that it comprises said zoledronic acid at a concentration between 0.5 and 25 mg/ml.

5. The pharmaceutical composition according to claim 1, characterized in that it comprises said zoledronic acid at a concentration between 4 and 16 mg/ml.

6. The pharmaceutical composition according to claim 1, characterized in that it further comprises a thickener selected from the group comprised by albumin and PEG

7. The pharmaceutical composition according to claim 1, characterized in that it further comprises a surfactant selected from the group comprised by sodium taurodeoxycholate, sodium taurocholate, sodium cholate, sodium deoxycholate, macrogol 15 hydroxystearate, sorbitan monolaurate, Tween, and mixtures thereof.

8. The pharmaceutical composition according to claim 7, characterized in that it comprises said surfactant at a concentration between 0.01 and 1% w/v.

9. The pharmaceutical composition according to claim 1, characterized in that said pH regulator is selected from the group comprised by sodium hydroxide, sodium phosphate, sodium citrate, sodium lactate, and mixtures thereof.

10. The pharmaceutical composition according to claim 1, characterized in that it further comprises a vasodilator selected from the group comprised by acetylcholine chloride and adenosine triphosphate, and their mixtures.

11. The pharmaceutical composition according to claim 10, characterized in that it comprises said vasodilator at a concentration between 0.01 and 2% w/v.

12. The pharmaceutical composition according to claim 1, characterized in that it further comprises a neutral excipient selected from the group comprised by lactose, sorbitol, sucrose, mannitol and their mixtures.

13. The pharmaceutical composition according to claim 12, characterized in that it comprises said excipient at a concentration between 0.01 and 8% w/v.

14. The pharmaceutical composition according to claim 1, characterized in that it further comprises an adjuvant excipient selected from the group comprised by aluminum hydroxide, zinc acetate and their mixtures.

15. The pharmaceutical composition according to claim 14, characterized in that it comprises said adjuvant excipient at a concentration between 0.01 and 1.8% w/v.

16. The pharmaceutical composition according to claim 1, characterized in that it comprises a pH between 6 and 7.4.

17. The pharmaceutical composition according to claim 1, characterized in that said solvent is selected from the group comprised by water, ethanol, propylene glycol, glycerol, glycofurol, isopropyl alcohol, dimethylsulfoxide and their mixtures.

18. The pharmaceutical composition according to claim 1, characterized in that said container is made of glass with an inner surface silicon dioxide film.

19. The pharmaceutical composition according to claim 1, characterized in that said container is made of improved type I glass sealed with a Teflon-coated bromobutyl stopper that can be sterilized by an autoclave.

20. The pharmaceutical composition according to claim 1, characterized in that said container is a syringe.

21. The pharmaceutical composition according to claim 1, characterized in that it comprises a liquid volume total between 0.1 and 100 ml.

22. The pharmaceutical composition according to claim 1, characterized in that its total final volume is between 0.2 and 5 ml.

23. The pharmaceutical composition according to claim 1, characterized in that its total final volume is between 0.25 and 2 ml.

24. The pharmaceutical composition according to claim 19, characterized in that after being sterilized it is essentially free of extractables and said stoppers continue to be hermetically sealed.
